(19) United States
(12) Patent Application Publication
(10) Pub. No.: US 2007/0149932 A1
(43) Pub. Date: Jun. 28, 2007

(54) Eye-drop dispensing eyecup

(76) Inventor: Robert Nick, Rancho Santa Margarita, CA (US)

Correspondence Address: Robert Nick, POB 3156, Laguna Hills, CA, 92654, US

(21) Appl. No.: 11/319,988

(22) Filed: Dec. 28, 2005

(51) Int. Cl. A61M 35/00; A61H 33/04

(52) U.S. Cl. 604/295; 604/289; 604/301

(57) ABSTRACT

The present invention relates to an eyecup that can be removably attached to the nozzle or neck of an eye-drop dispensing container to aid in placing eye-drops into the eye. The eyecup has an open top end and a base end with an aperture therethrough adapted to receive and support the nozzle or neck of the container.

### FIELD OF THE INVENTION

The invention relates to eyecups and more particularly to eyecups for administering eye-drops.

### BACKGROUND OF THE INVENTION

Ophthalmic solutions such as prescription drugs, red eye treatments, dry eye treatments and the like are typically introduced into the eye using an eyedropper or a squeeze-type container with a tapered tip or nozzle. Such containers are held above an eye to be treated.

The insertion of a foreign substance into the eye is unnatural and unnerving however and many people shake and/or blink as the drops are inserted into the eye. In addition, a significant number of people lack the strength to stabilize the dropper or squeeze container over the eye for the period of time required to insert the drops. Problems arise, such as bad aim, spillage, misdosage, contamination of the dropper tip and, for women, makeup damage. Also, it is important to accurately place the eye-drops into the eye to avoid waste of the medicine and to ensure an easier adjustment to such a foreign substance. As such, inserting eye-drops, medicines or solutions into the eye has been a long-time problem for many people.

Over the years, various devices have been developed to make the insertion of eye-drops easier. U.S. Pat. No. 4,531,944, for example, teaches the use of an eye-drop application aid that includes a housing that covers the eye. A dropper is then inserted into the housing and aligned over the pupil by a vertical alignment indicator. This device has removable dispenser seats that conform to the various dispenser tips of common eye-drop vessels. Such a device has several parts and is unnecessarily complicated and expensive to manufacture.

U.S. Pat. No. 6,508,793 teaches an eye-drop device having an eyecup and a threaded column for attachment to a threaded container. This device also has several parts and is unnecessarily complicated and expensive to manufacture.

What is needed is a simple, one piece device, that is especially useful for inserting eye-drops into the eye and that can be universally used with eye-drop solution containers.

### BRIEF SUMMARY OF THE INVENTION

The present invention relates to an eyecup adapted to aid in placing eye-drops into the eye. More specifically, the invention is an eyecup alone that can be removably attached to the nozzle or neck of an eye-drop dispensing container.

### BRIEF DESCRIPTION OF THE DRAWINGS

FIG. 1 is a perspective view of an eyecup of the present invention;

FIG. 2 illustrates an eyecup of the invention attached to the nozzle of an eye-drop dispensing container;

FIG. 3 illustrates an eyecup of the invention attached to the neck of an eye-drop dispensing container; and

FIG. 4 is a perspective view of a variable aperture eyecup of the invention.

### DETAILED DESCRIPTION OF THE INVENTION

A conventional eyecup, for administering ophthalmic solutions, has an open top end and a base end. In use, the eyecup is first filled with a solution and the open top end is then placed over the eye area to deliver the solution into the eye.

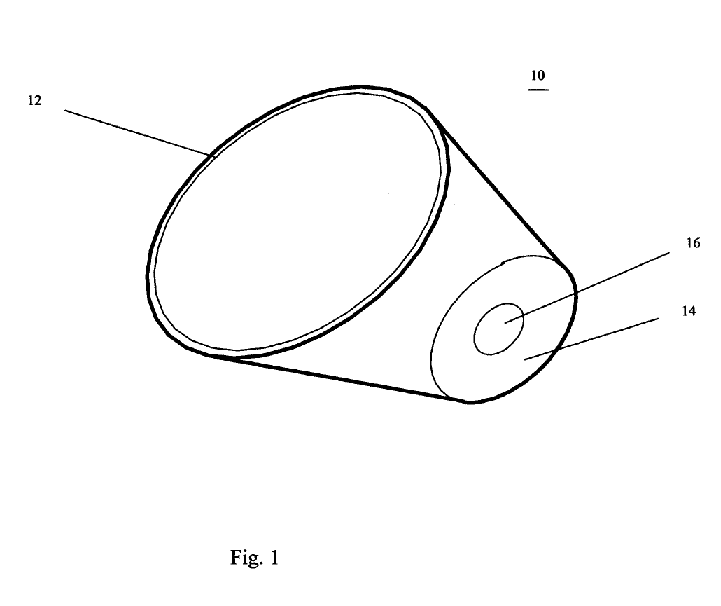

With reference to FIG. 1, an eyecup 10 of the invention has a base end 14 and an open top end 12 where the open top end can be positioned around a human eye area to aid in dispensing drops of an eye treatment solution into the eye. A preferred embodiment of the eyecup has a narrower cross section toward the base end, flaring out to a wider cross section away from the base end.

The base end of the eyecup has an aperture 16 therethrough adapted to receive and support the nozzle or neck of an eye-drop dispensing container. The aperture provides a means to place the nozzle into the eyecup without contaminating the nozzle tip.

The aperture may be circular or oval in shape but may be any shape adapted to accommodate different types of eye-drop dispensing container nozzles, including containers that have a nozzle with a threaded neck. Such containers may have a threaded neck adapted to secure a cap, for example, where the cap has a matching set of internal threads.

The eyecup is preferably made of transparent or translucent semi-rigid polymeric material but could be made of other materials such as glass, ceramic, metal, carbon fiber or the like.

Preferably, the eyecup has a truncated conical shape with an open top end adapted to provide relative conformity to the human and anatomical curvature about a human eye and a base end optimally spaced from the top end so that the nozzle or neck of an eye-drop dispensing container, inserted into the base end aperture, does not contact the eye or eyelash area, thereby permitting an eye-drop solution to be safely dispensed.

Although a conical structure is shown, it is understood that the eyecup but may be of any appropriate shape including concave, hemispherical, elliptical and curvilinear forms.

An important element of the invention is its simple unistructural form that is easy to use and that can be inexpensively manufactured as a one-piece unit.

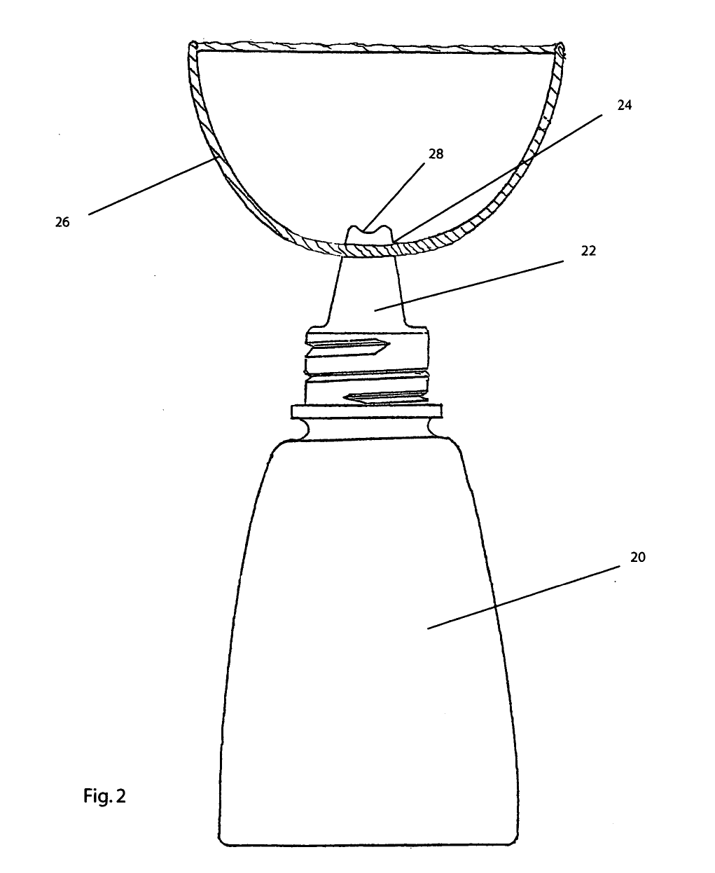

With reference to FIG. 2, the tip 28 of the nozzle 22 of an eye-drop dispensing container 20 is shown inserted into the base end aperture 24 of the eyecup housing 26. The aperture is configured so that the nozzle can be placed into the aperture without having the tip touch the eyecup housing. In use, the eyecup is placed over the eye and the container is squeezed to dispense the eye drops.

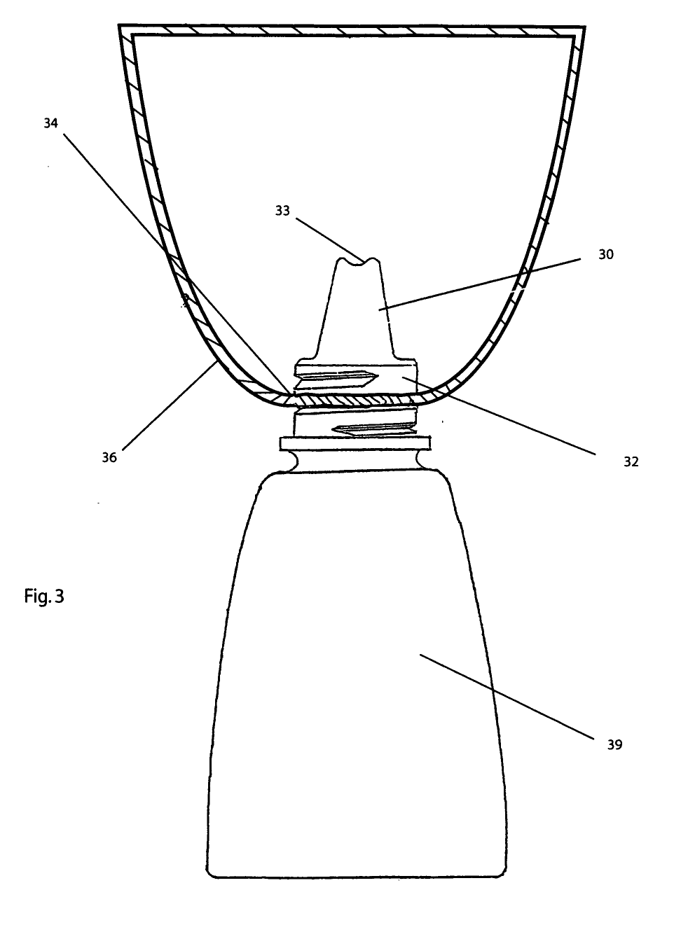

With reference to FIG. 3, the threaded neck portion 32 of a nozzle 30 of an eye-drop dispensing container 39 is shown inserted into a circular base end aperture 34 of the eyecup housing 36 of the invention.

In use, the container nozzle is inserted into the base end aperture and rotated so that the threaded neck portion of the nozzle engages the rim of the aperture to secure the container to the eyecup without requiring the need for a set of eyecup matching threads.

The eyecup is placed over the eye and the container is squeezed to dispense the eye drops.

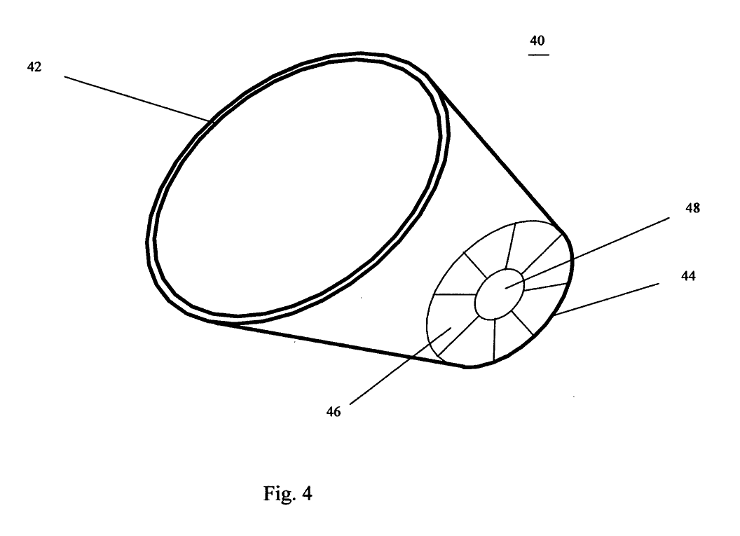

With reference to FIG. 4, a perspective view of an eyecup 40 of the invention is shown having an open top end 42 and a base end 44 configured with a plurality of flexible members 46 defining an aperture 48.

The flexible members are adapted to grip a dispenser container nozzle and secure the nozzle to the eyecup regardless of whether the nozzle is threaded or smooth. As such, various sizes of dispenser container nozzles can be accommodated with a single eyecup configuration.

Numerous modifications and variations of the present invention are possible in light of the above teachings. It is therefore understood that within the scope of the appended claims, the invention may be practiced otherwise than as specifically described herein.

What is claimed is:

1. Eyecup means for dispensing eye-drops into the eye, said eyecup means having a shape adapted to conform to the human and anatomical curvature about a human eye, said eyecup means further having an aperture therein adapted to receive the dispensing nozzle of an eye-drop solution container.

2. Eyecup means as in claim 1 wherein said eyecup is removable from said nozzle.

3. Eyecup means as in claim 1, wherein said eyecup shape is curvilinear.

4. Eyecup means as in claim 1, wherein said eyecup shape has a truncated conical form.

5. Eyecup means as in claim 1 wherein said eyecup is unistructural in form.

6. Eyecup means as in claim 1 wherein said aperture is circular in shape.

7. Eyecup means as in claim 1 wherein said dispensing nozzle has a threaded neck portion and wherein said threaded neck portion engages the rim of said aperture to secure said container to said eyecup.

8. Eyecup means as in claim 1 wherein said eyecup is made of polymeric material.

9. Eyecup means as in claim 1 wherein said eyecup is made of semi-rigid material.

10. Eyecup means for dispensing eye-drops into the eye, said eyecup means having an open top end and a base end, said open top end adapted to conform to the human and anatomical curvature about a human eye, said base end having an aperture therethrough adapted to receive the dispensing nozzle of an eye-drop solution container.

11. Eyecup means as in claim 10 wherein said eyecup is removable from said nozzle.

12. Eyecup means as in claim 10, wherein said eyecup shape is curvilinear.

13. Eyecup means as in claim 10, wherein said eyecup shape has a truncated conical form.

14. Eyecup means as in claim 10 wherein said eyecup is unistructural in form.

15. Eyecup means as in claim 10 wherein said aperture is circular in shape.

16. Eyecup means as in claim 10 wherein said dispensing nozzle has a threaded neck portion and wherein said aperture is adapted to receive the threaded neck portion of said dispensing nozzle, said threaded neck portion engaging the rim of said aperture to secure said container to said eyecup.

17. Eyecup means as in claim 10 wherein said eyecup has a narrower cross-section toward said base end flaring out to a wider cross-section away from said base end.

18. Eyecup means as in claim 10 wherein said eyecup is made of polymeric material.

19. Eyecup means as in claim 10 wherein said eyecup is made of semi-rigid material.

20. Eyecup means as in claim 10 wherein said base end has a plurality of flexible members defining said aperture, said flexible members adapted to grip said dispensing nozzle and to secure said nozzle to said eyecup.
